(19) United States
(12) Patent Application Publication
(10) Pub. No.: US 2017/0007216 A1
(43) Pub. Date: Jan. 12, 2017

(54) ENDOCYTE CANNULA

(71) Applicant: All Cape Gynecology LLC, Hyannis, MA (US)

(72) Inventor: Lucia Cagnes, Sandwich, MA (US)

(21) Appl. No.: 15/117,994

(22) PCT Filed: Feb. 11, 2015
(86) PCT No.: PCT/US15/15449

§ 371 Date: Aug. 10, 2016

Related U.S. Application Data
(60) Provisional application No. 61/938,377, filed on Feb. 11, 2014.

(51) Int. Cl. A61B 10/02; A61M 1/00

(52) U.S. Cl. CPC A61B 10/0291; A61M 1/0009; A61B 2010/009

(57) ABSTRACT

An endocyte cannula is designed to provide improved and more productive Pap smear tests. The endocyte cannula includes a proximal portion and a distal portion. The proximal portion may include a fitting to connect to a user-actuated tool, such as a syringe. The distal portion may include a corkscrew feature to facilitate movement of the cannula through dense tissue. The distal portion may further include a plurality of apertures to aspirate mucus and reduce the risk of trauma or perforation.

### CROSS-REFERENCE TO RELATED APPLICATIONS

This application claims priority to U.S. Provisional Application No. 61/938,377 entitled “ENDOCYTE CANNULA,” filed on Feb. 11, 2014, which is hereby incorporated by reference in its entirety.

### FIELD OF INVENTION

The invention pertains to an endocyte cannula for use in Pap smears and other medical related tests.

### BACKGROUND OF THE INVENTION

Papanicolaou tests (“Pap tests” or Pap smears) are commonly performed tests that have been proven to be highly valuable in the early detection of cervical pre-cancerous and cancerous growths. The Pap test refers to the collection of cells from the cervical face, the endocervical canal, and occasionally from the vaginal wall. The collected cells are subsequently “smeared” onto a microscope plate or deposited and mixed into a broth and analyzed for evidence of pre-cancerous or cancerous growth. A periodic Pap test permits the early detection of malignant cells, which enables the treatment of cervical pre-cancerous and cancerous growths.

Numerous devices have been developed to assist in the collection of cells during a Pap test. One such device is a wooden or plastic spatula. Such spatulas are inexpensive and can be effective at collecting cells from the cervical face. However, spatulas have proven to be less than effective in collecting adequate cell samples from the endocervical canal. This is a potentially serious short-coming, because any sample that does not include endocervical cells is deemed to be an inadequate Pap smear sample. That is to say, the proper interpretation and diagnosis of the state of the cells is inconclusive unless a sufficient number of cells are collected from the endocervical canal.

Other devices that are useful in collecting cells during Pap tests include certain bristle brushes in conjunction with a spatula. In this regard, the bristle brushes are capable of obtaining endocervical cells during sampling, however bristle brushes are not always able to penetrate a narrow endocervical canal that may be completely closed in a give patient.

Pap tests have proven to be useful in the early detection of malignant cells and are related to a reduction in the incidence and death rate due to cervical cancers. Improvements to sampling devices useful in collecting cells during Pap tests will be welcomed by the medical community and patients alike. Therefore, there is a need for an improved sampling device that is able to overcome at least one of the deficiencies noted above regarding other sampling devices.

### SUMMARY OF THE INVENTION

An endocyte cannula is generally provided. The endocyte cannula is designed to provide improved and more productive Pap smear tests. The endocyte cannula includes a proximal portion and a distal portion. The proximal portion may include a fitting to connect to a user-actuated tool, such as a syringe. The distal portion may include a corkscrew feature to facilitate movement of the cannula through dense tissue. The distal portion may further include a plurality of holes to aspirate mucus and reduce the risk of trauma or perforation to the patient.

In one embodiment, the present technology includes a medical device having a hollow tubular body having a proximal end and a distal end, wherein the proximal end is configured to connect to a user-control mechanism.

In one aspect, the distal end of the hollow tubular body includes a corkscrew-shaped portion having at least one spiral.

In one aspect, the distal end includes at least one aperture, the aperture having a first opening through an outer wall of the medical device and a second opening through the hollow tubular body.

In one aspect, the at least one aperture is configured to aspirate bodily tissues.

In one aspect, the proximal end includes a fitting configured to connect to the user-control mechanism.

In one aspect, the fitting is a luer lock.

In one aspect, the user-control mechanism is a syringe.

In one aspect, the syringe interfaces with the hollow tubular body to create a force through the medical device.

In one aspect, the hollow tubular body is configured to be received into a body cavity.

In one aspect, the hollow tubular body is configured to collect cells from a body cavity.

In one aspect, the body cavity is an endocervical canal.

In one aspect, the hollow tubular body comprises steel, stainless steel, plastic, rubber, or a combination of two or more thereof.

In one embodiment, the present technology provides a method for completing a Papanicolaou tests includes inserting a medical device into a endocervical canal. The medical device includes a hollow tubular body having a proximal end and a distal end, wherein the proximal end connects to a user-control mechanism and the distal end includes a corkscrew-shaped portion comprising at least one spiral and at least one aperture. Cells are collected from the endocervical canal with the medical device.

In one aspect, the corkscrew-shaped portion advances through bodily tissue.

In one aspect, the apertures aspirate bodily tissues.

In one embodiment, the present technology provides a kit for gathering endocervical canal tissue. The kit includes a medical device having a hollow tubular body with a proximal end and a distal end, wherein the proximal end is configured to connect to a user-control mechanism. The kit also includes a user-control mechanism.

In one aspect, the distal end of the medical device further comprises a corkscrew-shaped portion comprising at least one spiral and at least one aperture.

In one aspect, the user-control mechanism is selected from a manually actuated tool, a robot actuated tool, or an automated tool, or any combination of two or more thereof.

### BRIEF DESCRIPTION OF THE DRAWINGS

The invention may be better understood by reference to the following detailed description taken in connection with the following illustrations, wherein:

FIG. 1 is a plan view of an endocyte cannula.

FIG. 2 is a cross-sectional view of the endocyte cannula of FIG. 1 along the line 2.

FIG. 3 is a perspective view of a different embodiment of an endocyte cannula.

FIG. 4 is a perspective view of the endocyte cannula selectively secured to a syringe.

FIG. 5 is a perspective view of the endocyte cannula selectively secured to a syringe.

FIG. 6 is a perspective view of the fitting of the endocyte cannula.

### DETAILED DESCRIPTION OF THE INVENTION

Reference will now be made in detail to exemplary embodiments of the present invention, examples of which are illustrated in the accompanying drawings. It is to be understood that other embodiments may be utilized and structural and functional changes may be made without departing from the respective scope of the invention. Moreover, features of the various embodiments may be combined or altered without departing from the scope of the invention. As such, the following description is presented by way of illustration only and should not limit in any way the various alternatives and modifications that may be made to the illustrated embodiments and still be within the spirit and scope of the invention.

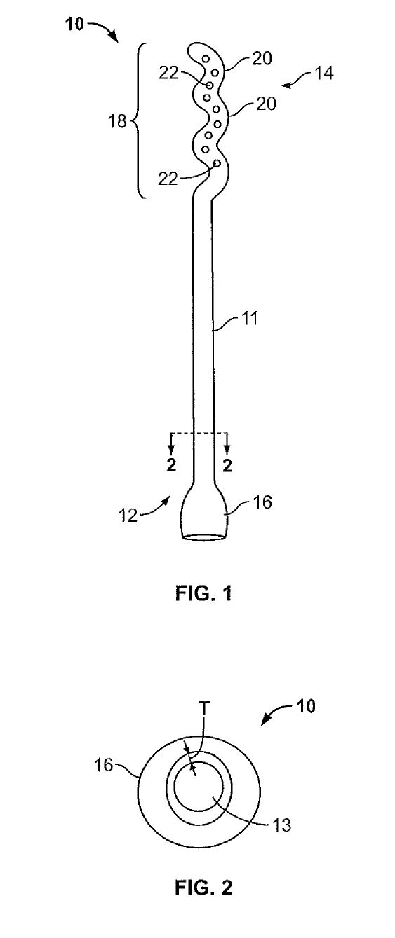

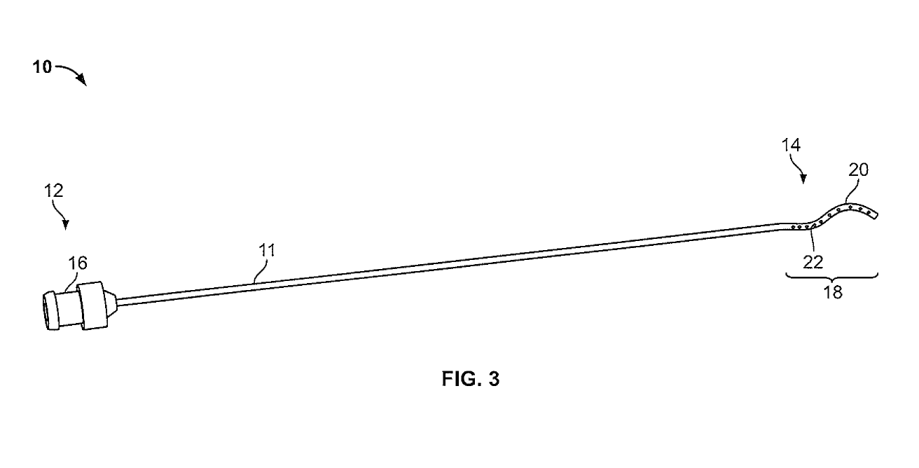

An endocyte cannula 10 is depicted in FIGS. 1 and 3. The endocyte cannula 10 may be configured for use in Pap smear tests and other medical related tests. As described below and illustrated in the Figures, the endocyte cannula 10 may improve the results and method of conducting a Pap smear. While the endocyte cannula 10 is shown and described in use with a Pap smear, the present teachings are not limited to such. The endocyte cannula 10 may be utilized in any appropriate medical related testing.

FIG. 1 depicts the endocyte cannula 10. The endocyte cannula 10 may be comprised of any appropriate material, including, without limitation, steel, stainless steel, plastic, rubber, a combination of the foregoing or any other appropriate material. The endocyte cannula 10 may include a body 11. The body 11 may be generally hollow and may include any appropriate wall thickness, such as T in FIG. 2. T may be any appropriate thickness, including for example, 0.1 mm-10 mm. As shown in FIG. 2, the body 11 may include a generally hollow tubular portion 13, which may be used in the aspiration process described in more detail below.

The endocyte cannula 10 may include a proximal end 12 and a distal end 14. The proximal end 12 may be generally configured to connect to a user-control mechanism. By way of a non-limiting example, the user control mechanism may include a syringe 15 or the like. The distal end 14 may be designed to generally increase effectiveness of use and allow for aspiration, as further described below.

The endocyte cannula 10 may be any appropriate size and shape. By way of a non-limiting example, the endocyte cannula 10 may be approximately 12 cm in total length. It will be appreciated, however, that then length of the endocyte cannula 10 may be longer or shorter than 12 cm, and may be any appropriate length. For example, the endocyte cannula 10 may be 5 cm to 50 cm in length. The diameter of the endocyte cannula 10 may be similar to that of a 20 gauge needle, such as between 0.6 mm and 1.0 mm. Alternatively, the diameter of the endocyte cannula 10 may be greater or smaller than that of a 20 gauge needle, for example, the diameter may range from 0.1 mm to 10 mm. The diameter may be consistent throughout the entirety of the endocyte cannula 10 or may vary across the length or at different portions of the endocyte cannula 10.

The proximal end 12 may be configured to connect to a user control mechanism. For example, the proximal end 12 may include a fitting 16, such as a taper or other feature, to interface with the syringe 15. In an embodiment, the fitting 16 may comprise a “luer taper” or “luer lock” or any other appropriate type of fitting or means or connection to provide an interface between the endocyte cannula 10 and the syringe 15 or other tool. While the syringe 15 is shown as being used with the endocyte cannula 10, it should be understood that any appropriate tool may be used and the teachings are not limited to the syringe 15 shown and described.

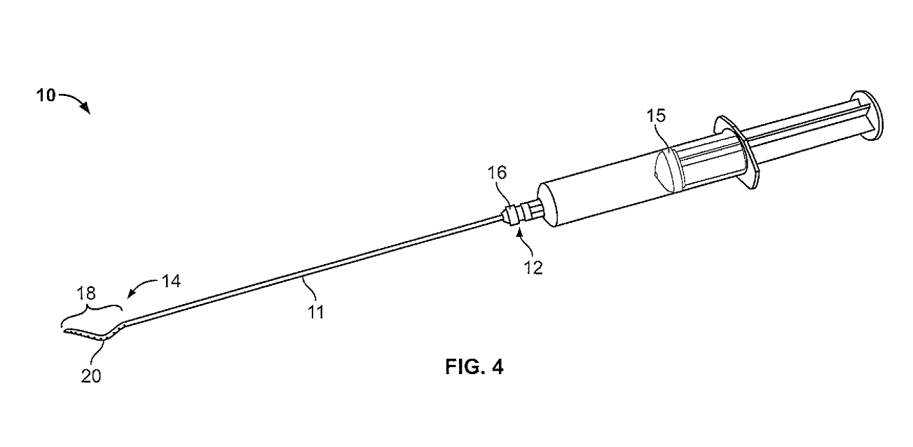

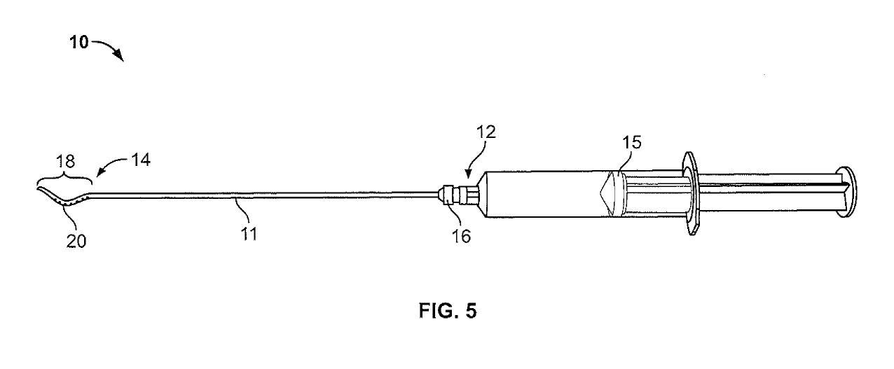

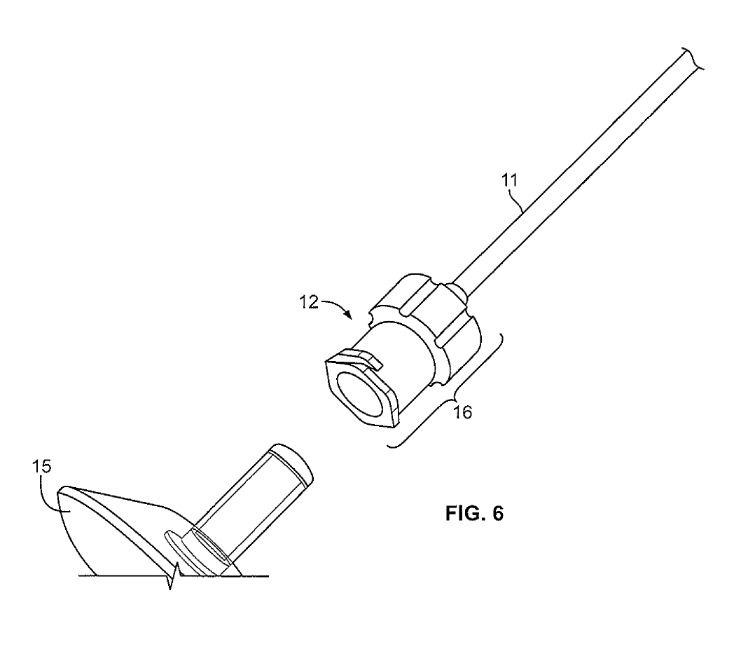

In some embodiments, the syringe 15 may be selectively attached to the fitting 16, as shown in FIGS. 4-6. The syringe 15 may interface with the generally hollow endocyte cannula 10, specifically, the tubular portion 13 to apply a force, pressure or suction therethrough. While the endocyte cannula 10 is described as connected to the syringe 15, it will be appreciated that the proximal end 12 may be connected to any appropriate tool, including any manually actuated, robot actuated or automated tool.

The distal end 14 may be configured to enhance the functionality and use of the endocyte cannula 10. By way of a non-limiting example, the distal end 14 may include a generally corkscrew shaped portion 18, as generally illustrated in FIG. 1. The corkscrew portion 18 may be any appropriate size, shape and length, such as approximately 25 mm in length. However, the corkscrew portion 18 may alternatively be longer or shorter than 25 mm, as appropriate. For example, the corkscrew portion may be 1 mm to 50 mm.

The corkscrew portion 18 may comprise one or more spirals 20. The spirals 20 may have any appropriate radius. In some embodiments, the radius of the spirals 20 may be approximately 3 mm. The radius of all spirals 20 may be generally consistent, or may vary along the length of the corkscrew portion 18.

The distal end 14 or more specifically the corkscrew portion 18 may include a plurality of apertures 22 disposed there along. The apertures 22 may assist the endocyte cannula 10 in advancing through dense tissue in the endocervical canal while at the same time minimizing the risk of perforation to the patient. The apertures 22 may aspirate mucus, thereby minimizing the risk of trauma to the cervical mucosa.

The apertures 22 may be formed through an outer wall of the endocyte cannula 10 and may open into the hollow opening of the endocyte cannula 10, i.e., the tubular portion 13. The apertures 22 may be arranged along the length of the corkscrew portion 18, or along other portions of the endocyte cannula 10. The distal end 14 may include any appropriate number of apertures 22 and are not limited to the number shown in FIGS. 1-6. Further, the apertures 22 may be positioned in any appropriate configuration. By way of a non-limiting example, the apertures may generally be evenly spaced from one another along the corkscrew portion 18. The apertures may generally be unevenly spaced from one another along the corkscrew portion 18. The apertures may be uniform in size or they may vary in size. The apertures may be round in shape, ovoid in shape, rectangular in shape, or any other appropriate shape.

The endocyte cannula 10 may also include other features, for example, bristles, rough surfaces, smooth surfaces, reflective surfaces, or matte surfaces. The endocyte cannula 10 may also include a light, a camera, or any other feature beneficial to the operator.

Although the embodiments of the present invention have been illustrated in the accompanying drawings and described in the foregoing detailed description, it is to be understood that the present invention is not to be limited to just the embodiments disclosed, but that the invention described herein is capable of numerous rearrangements, modifications and substitutions without departing from the scope of the claims hereafter. The claims as follows are intended to include all modifications and alterations insofar as they come within the scope of the claims or the equivalent thereof.

1. A medical device comprising:
a hollow tubular body having a proximal end and a distal end, wherein the proximal end is configured to connect to a user-control mechanism.

2. The medical device of claim 1, wherein the distal end of the hollow tubular body further comprises a corkscrew-shaped portion comprising at least one spiral.

3. The medical device of claim 2, wherein the distal end further comprises at least one aperture, the aperture having a first opening through an outer wall of the medical device and a second opening through the hollow tubular body.

4. The medical device of claim 3, wherein the at least one aperture is configured to aspirate bodily tissues.

5. The medical device of claim 4, wherein the proximal end further comprises a fitting configured to connect to the user-control mechanism.

6. The medical device of claim 6, wherein the fitting comprises a luer lock.

7. The medical device of claim 6, wherein the user-control mechanism is a syringe.

8. The medical device of claim 7, wherein the syringe interfaces with the hollow tubular body to create a force through the medical device.

9. The medical device of claim 8, wherein the hollow tubular body is configured to be received into a body cavity.

10. The medical device of claim 9, wherein the corkscrew-shaped portion is configured to collect cells from a body cavity.

11. The medical device of claim 10, wherein the body cavity is an endocervical canal.

12. The medical device of claim 11, wherein the hollow tubular body comprises steel, stainless steel, plastic, rubber, or a combination of two or more thereof.

13. A method for completing a Papanicolaou test comprising:
inserting a medical device into an endocervical canal,
the medical device comprising a hollow tubular body having a proximal end and a distal end, wherein the proximal end connects to a user-control mechanism and the distal end comprises a corkscrew-shaped portion comprising at least one spiral and at least one aperture; and
collecting cells from the endocervical canal with the medical device.

14. The method of claim 13, wherein the corkscrew-shaped portion advances through bodily tissue.

15. The method of claim 14, wherein the apertures aspirate bodily tissues.

16. A kit for gathering endocervical canal tissue comprising:
a medical device comprising a hollow tubular body having a proximal end and a distal end, wherein the proximal end is configured to connect to a user-control mechanism; and
a user-control mechanism.

17. The kit of claim 16, wherein the user-control mechanism is configured to connect to the proximal end of the medical device via a luer lock connection.

18. The kit of claim 17, wherein the user-control mechanism is selected from a manually actuated tool, a robot actuated tool, an automated tool, or any combination of two or more thereof.

19. The kit of claim 18, wherein the distal end of the medical device further comprises a corkscrew-shaped portion comprising at least one spiral and at least one aperture.

20. The kit of claim 19, wherein the user-control mechanism is a syringe.
